(19) United States
(12) Patent Application Publication
(10) Pub. No.: US 2019/0021706 A1
(43) Pub. Date: Jan. 24, 2019

(54) TISSUE COLLECTION DEVICE

(71) Applicant: TERUMO KABUSHIKI KAISHA, Tokyo (JP)

(72) Inventor: Kouichirou YORI, Kanagawa (JP)

(73) Assignee: TERUMO KABUSHIKI KAISHA, Tokyo (JP)

(21) Appl. No.: 16/137,792

(22) Filed: Sep. 21, 2018

Related U.S. Application Data
(63) Continuation of application No. PCT/JP2017/011624, filed on Mar. 23, 2017.

(30) Foreign Application Priority Data
Mar. 23, 2016 (JP) 2016-058142

(51) Int. Cl. A61B 10/02

(52) U.S. Cl. CPC A61B 10/0266

(57) ABSTRACT

A tissue collection device including a sheath member that has a first threaded portion on the inner surface of the sheath member and a puncture tube positionable in the interior of the sheath member. The puncture tube is axially movable in distal and proximal directions relative to the sheath member. The puncture tube has a second threaded portion on the outer surface of the puncture tube that threadedly engages with the first threaded portion when the puncture tube is within the interior of the sheath member. At least one of the distal end of the sheath member and the distal end of the puncture tube includes a blade to incise tissue from a living body.

### CROSS-REFERENCES TO RELATED APPLICATIONS

This application is a continuation of International Application No. PCT/JP2017/011624 filed on Mar. 23, 2017, and claims priority to Japanese Application No. 2016-058142 filed on Mar. 23, 2016, the entire content of both of which is incorporated herein by reference.

### TECHNICAL FIELD

The present invention generally relates to a device configured to perform tissue collection quantitatively (a device configured to collect a predetermined amount of tissue without relying on manual operation).

### BACKGROUND DISCUSSION

Regenerative medical products are obtained by performing a process such as cultivation for human cells using a human tissue or the like as a raw material. In order to produce regenerative medical products, it is necessary to secure a certain amount of tissue for the regenerative medical product. The human body is thus incised by a scalpel or the like to collect the tissue. However, this type of method is relatively high in invasiveness and has a relatively large impact on the human body.

A tissue collection needle for biopsy or the like is available as a relatively low invasive means. However, the tissue collection needle or the like is for collecting a (relatively) very small amount of tissue for inspection, and it is necessary to use such a needle as described above multiple times in the human body in order to secure an amount of tissue necessary for a regenerative medical product.

For example, Japanese Patent Application Publication No. 2000-60859 discloses a cell tissue collector that includes a tubular body having a distal portion formed in a shape of a needle of a syringe and a brush bar that possesses a distal portion formed in a shape of a needle and has brush hair provided in a projecting manner in the proximity of the distal portion. The brush bar of this cell tissue collector fits into the tubular body such that the brush hair and the distal needle are moved into and out of the tubular body to collect cells from a wide range in an internal organ. The cell tissue collector has a problem, however, in being relatively high in invasiveness or the like in the human body.

Japanese Patent Application Publication No. 2012-235878 discloses a biopsy device that includes a tube shaped sheath and a core shaft configuring a needle main body having a spiral groove at a distal portion of the needle main body. According to this biopsy device, the core shaft is moved into and out of the sheath to collect tissue into the spiral groove. However, this biopsy device has a relatively small collection amount because the core shaft occupies the volume in the sheath.

Japanese Patent Application Publication No. 2015-85141 discloses a puncture needle that includes a sheath member, a needle tube, and a needle member having a rotary blade. Graduations are provided on a needle member operation unit and a needle tube operation unit. The puncture needle is configured so that the operator adjusts the length over which the needle member projects while viewing the graduations. However, since such adjustment relies upon the manipulation of the operator, such a problem that tissue cannot be collected quantitatively or the like is concerned.

### SUMMARY

The inventor of the present invention has identified problems such as a conventional biopsy device that collects tissue through back and forth movement of a needle member cannot secure a sufficient amount of tissue by a single operation, that there is a difference in collection amount depending upon the manipulation of the operator, and so forth. The device of this application may help address these problems by collecting tissue quantitatively (i.e., of a predetermined amount) by a simple operation.

The inventor has conducted intensive research in order to develop a device with which any person can quantitatively collect tissue by a simple operation while preventing differences in collection amounts due to different manipulations by the operator (i.e., a device that requires manual operation to control the amount of tissue collected may result in differing amounts of tissue collected in separate uses). The inventor has found that tissue can be collected quantitatively by controlling forward and backward movement of a tissue collection device by a threaded engagement mechanism.

In particular, the tissue collection device disclosed in this application relates to the following.

In one aspect, the disclosure relates to a tissue collection device including a sheath member that has a first threaded portion on the inner surface of the sheath member and a puncture tube positionable in the interior of the sheath member. The puncture tube is axially movable in distal and proximal directions relative to the sheath member. The puncture tube has a second threaded portion on the outer surface of the puncture tube that threadedly engages with the first threaded portion when the puncture tube is within the interior of the sheath member. At least one of the distal end of the sheath member and the distal end of the puncture tube includes a blade to incise tissue from a living body.

In another aspect, the disclosure involves a tissue collection device for collecting tissue in a living body. The tissue collection device includes a sheath member comprising a main body and a first threaded portion. The sheath member extends in an axial direction from a distal end to a proximal end. The first threaded portion of the sheath member is proximal to the main body. The sheath member is a tubular body with the distal and proximal ends being open. The first threaded portion is on the inner surface of the sheath member. The sheath member possesses an outer surface, an inner surface and an inner diameter. A puncture tube is positionable in the sheath member, the puncture tube extending in the axial direction from a distal end to a proximal end. The puncture tube includes a blade at the distal end of the puncture tube and a second threaded portion proximal to the blade. The second threaded portion is on the outer surface of the puncture tube. The puncture tube possesses an outer surface, an inner surface and an outer diameter. The second threaded portion on the outer surface of the puncture tube is configured to threadedly engage the first threaded portion on the inner surface of the sheath member when the puncture tube is positioned in the sheath member so that relative rotation between the sheath member and the puncture tube in one rotational direction axially moves the blade into the tissue in the living body to incise an amount of tissue, and so that relative rotation between the sheath member and the puncture tube in the opposite rotation direction axially moves the blade out of the living body with the incised tissue held at the distal end of the puncture tube. The inner diameter of the main body of the sheath member is smaller than the outer diameter of the second threaded portion of the puncture tube, such that the second threaded portion of the puncture tube is prevented from moving distally into the main body of the sheath member.

In yet another aspect, the disclosure involves a method for extracting a predetermined amount of tissue from a living body. The method includes contacting the living body with a distal end of a sheath member and axially moving a puncture tube positioned inside the sheath member in a distal direction relative to the sheath member to cause a distal end of the puncture tube to contact the tissue in the living body. The axially moving of the puncture tube relative to the sheath member occurring by virtue of a threaded portion of the puncture tube threadedly engaging a threaded portion of the sheath member. The method includes incising the predetermined amount of the tissue in the living body while axially moving the puncture tube in the distal direction and automatically stopping the axial movement of the puncture tube relative to the sheath member in the distal direction after the predetermined amount of the tissue has been incised. The method includes extracting the predetermined amount of the tissue from the living body.

[4] The tissue collection device according to any one of aforesaid [1] to [3], in which the puncture tube includes an outer side tubular body and an inner side tubular body fitted for back and forth movement in the outer side tubular body.

[5] The tissue collection device according to aforesaid [4], in which the inner side tubular body has a groove extending in an axial direction thereof.

[6] The tissue collection device according to any one of aforesaid [1] to [5], further including:

a vessel for accommodating the puncture tube, the vessel having a third threaded portion for threaded engaging with the second threaded portion.

[7] The tissue collection device according to any one of aforesaid [1] to [6], in which at least part is configured from an optically transparent material.

The device of this application can quantitatively collect tissue by a simple operation. Further, by using the device disclosed here, tissue can be quantitatively collected by a single time operation without depending upon the manipulation of the operator, and therefore, relatively low invasiveness in the living body (i.e., patient) can be achieved.

### BRIEF DESCRIPTION OF THE DRAWINGS

FIG. 1 is a sectional view schematically depicting a device according to a first embodiment.

FIGS. 2A to 2D are sectional views schematically depicting an example use of the device shown in FIG. 1.

FIGS. 3A and 3B are sectional views schematically depicting a spiral blade according to an embodiment.

FIG. 4 is a sectional view schematically depicting a device according to a second embodiment.

FIG. 5 is a sectional view schematically depicting a device according to a third embodiment.

FIGS. 6A and 6B are sectional views schematically depicting an example use of the device shown in FIG. 5.

FIGS. 7A and 7B are sectional views schematically depicting a vessel of the present invention.

### DETAILED DESCRIPTION

Set forth below with reference to the accompanying drawings is a detailed description of embodiments of a tissue collection device and a method of tissue collection representing examples of the inventive tissue collection device and method disclosed here. Note that the size of each member (i.e., component) in the figures is suitably emphasized for illustration purposes and does not necessarily correlate to an actual ratio or magnitude. In the following description, the term “proximal portion” and “proximal end” signifies an end portion or end of the device on the operator side, and the term “distal portion” and “distal end” signifies an end portion or end of the device on the tissue side (i.e., in the living body during tissue collection).

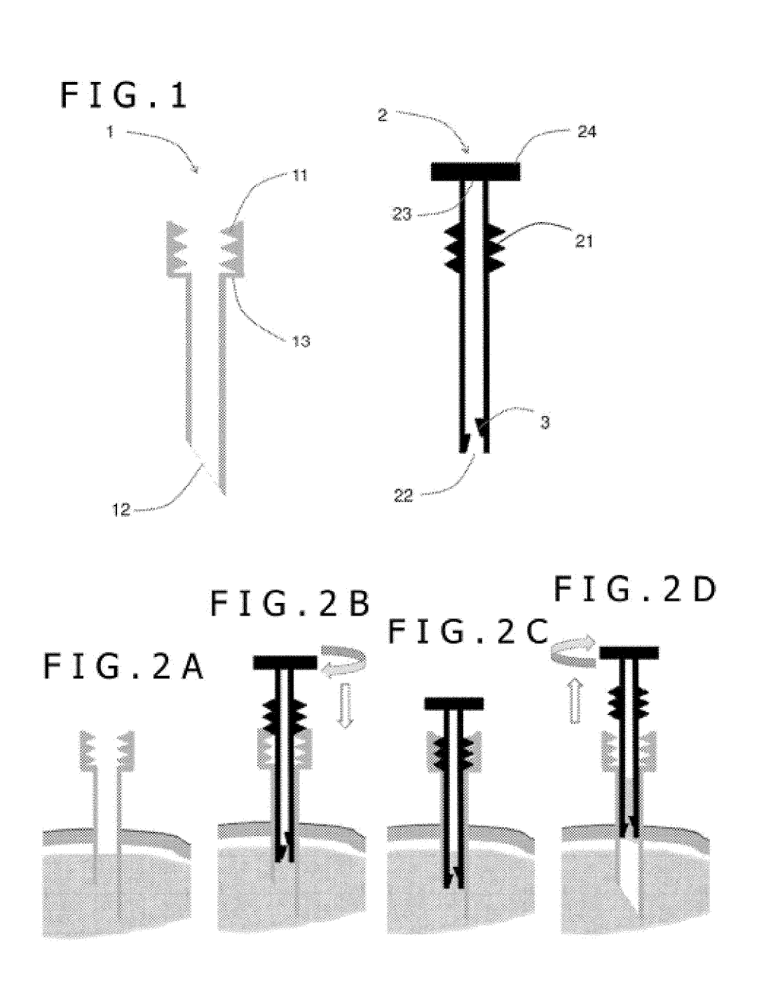

A first embodiment of a device disclosed in this application is first described in relation to FIG. 1. FIG. 1 is a sectional view schematically depicting the device according to the first embodiment. The device includes a sheath member 1 (i.e., a sheath) and a puncture tube 2. The puncture tube 2 can be fitted for back and forth movement in the sheath member 1 (i.e., the puncture tube 2 is configured to axially move distally and proximally within the sheath member 1 relative to the sheath member 1). Both the sheath member 1 and the puncture tube 2 have a form of a tubular body (i.e., the sheath member 1 and the puncture tube 2 are each tubular). The sheath member 1 has a first threaded portion 11 provided in a projecting manner on an inner face on the proximal side of the sheath member 11 (i.e., the first threaded portion 11 extends radially inward from the inner surface of the sheath member 1). The sheath member 1 includes a distal portion 12 formed as a blade of a pointed shape that is inclined with respect to a plane perpendicular to a long axis (longitudinal axis) of the sheath member 1. The distal portion 12 can thus pierce the tissue. The puncture tube 2 has a second threaded portion 21 provided in a projecting manner on an outer face on the proximal side of the puncture tube 21 (i.e., the second threaded portion 21 extends radially outward from the outer surface of the puncture tube 2) for threadedly engaging with (i.e., threaded engagement with) the first threaded portion 11. The length of the first threaded portion 11 is substantially equal to the length of the second threaded portion 21, and the length of the distal side of the sheath member 1 with respect to the first threaded portion 11 is substantially equal to the length of the distal side of the puncture tube 2 with respect to the second threaded portion 21 (i.e., the length of the sheath member 1 that is distal to the first threaded portion 11 is the same or about the same as the length of the puncture tube 2 that is distal to the second threaded portion 21).

A spiral blade 3 is provided in a projecting manner on the inner face at a distal portion 22 of the puncture tube 2 (i.e., the spiral blade 3 extends radially inward from the inner surface of the puncture tube 2 at a distal portion 22 of the puncture tube 2). An operation unit 24 (e.g., a plunger) is provided at a proximal portion 23 of the puncture tube 2. The outer diameter of the proximal side of the sheath member 1 on which the first threaded portion 11 is provided is greater than the outer diameter of the sheath member 1 distal to the first threaded portion 11. A stopper 13 is formed by this offset (i.e., difference) between the outer diameters as shown in FIG. 1. The inner diameter of the sheath member 1 distal to the first threaded portion 11 is smaller than the outer diameter of the second threaded portion 21 of the puncture tube 2 such that the second threaded portion 21 is not engaged with the distal side farther than the first threaded portion 11 of the sheath member 1 (i.e., the second threaded portion 21 is prevented from moving within the sheath member 1 distally beyond the distal end of the first threaded portion 11).

FIGS. 2A to 2D are sectional views schematically depicting an example use of the device according to the first embodiment that is shown in FIG. 1. The distal portion 12 of the sheath member 1 first punctures an application region of the living body to perform incision and cutting out of the tissue and takes the tissue (i.e., gathers or collects the tissue) into the inner side of the sheath member 1 (FIG. 2A). Since the sheath member 1 has the stopper 13, such a problem that the sheath member 1 inadvertently enters the application region, or a similar problem, is less likely to occur. The sheath member 1 may have graduations on an outer face of the sheath member 1 such that the puncture depth of the sheath member 1 at the application region can be confirmed by the operator.

The puncture tube 2 is then inserted into the sheath member 1 until the second threaded portion 21 of the puncture tube 2 and the first threaded portion 11 of the sheath member 1 are threadedly engaged with each other and the operation unit 24 is operated (i.e., rotated) to rotate the puncture tube 2 (as shown in FIG. 2B). While the puncture tube 2 rotates, it is threadedly engaged in a direction toward the distal end of the sheath member 1 (i.e., in a downward arrow mark direction of FIG. 2B) so that the puncture tube 2 moves distally or in a distal direction relative to the sheath member 1. The puncture tube 2 thus progressively collects the tissue in the sheath member 1 into the inner side of the puncture tube 2 while the spiral blade 3 of the puncture tube 2 shreds the tissue in the sheath member 1. The puncture tube 2 is preferably rotated in this manner while the sheath member 1 is fixed relative to the living body (i.e., puncture site).

The threaded engagement of the puncture tube 2 is stopped in the middle (i.e., in a fully engaged position between the first and second threaded portions 11, 21) as shown in FIG. 2C because the inner diameter of the distal side of the sheath member 1 is smaller than the outer diameter of the second threaded portion 21 of the puncture tube 2. In particular, even if the operator attempts to operate (i.e., rotate) the operation unit 24 further, the puncture tube 2 does not rotate and does not advance distally relative to the sheath member 1. When the tissue is to be taken out (i.e., removed or extracted), either the sheath member 1 is pulled out in a direction of an upward arrow mark together with the puncture tube 2 or the puncture tube 2 is rotated in the opposite direction to pull out the puncture tube 2 in the direction of the upward arrow mark as shown in FIG. 2D (i.e., proximally relative to the sheath member 1). When the puncture tube 2 is rotated to be pulled out (i.e., moved proximally relative to the sheath member 1), the sheath member 1 and the puncture tube 2 may be rotated integrally in advance such that the tissue in the puncture tube 2 is cut away with certainty (i.e., a predetermined amount of tissue is collected) from the application region by the spiral blade 3. Here, the threaded engagement between the sheath member 1 and the puncture tube 2 may be structured such that, if the puncture tube 2 is threadedly engaged to a fixed position in the sheath member 1, then the puncture tube 2 begins to idly rotate and does not retract proximally farther in the axial direction (i.e., when the puncture tube 2 moves proximally relative to the sheath member 1 such that the first and second threaded portions 11, 21 no longer engage with one another, the puncture tube 2 rotates relative to the sheath member 1 but does not move proximally against the first threaded portion 11). When the puncture tube 2 idly rotates in the sheath member 1 in this manner, the puncture tube 2 (i.e., via the spiral blade 3) can cut away the tissue of the living body. The tissue can thereby be collected with relatively lower invasiveness than that the invasiveness of an alternative case in which the puncture tube 2 is rotated together with the sheath member 1 to cut away the tissue.

As described above, a problem that the puncture tube 2 inadvertently enters the application region of the living body (i.e., puncture site) or a similar problem is less likely to occur because the disclosed device controls the movement of the puncture tube 2 in the distal and proximal directions using threaded engagement (i.e., via the first and second threaded portions 11, 21) with the sheath member 1. Further, since the distance of the back and forth movement of the puncture tube 2 depends upon the distance of rotation of the operation unit 24, for example, the collection amount of tissue can be adjusted quantitatively by the number of rotations of the operation unit 24. In other words, a predetermined amount of tissue may be collected based on a predetermined number of rotations of the operation unit 24. The stopper 13 on the outer face of the sheath member 1 may make the sheath member 1 inadvertently entering the application region (or a similar problem) relatively less likely to occur when the sheath member 1 punctures the application region of the living body.

Since the puncture tube 2 of the present invention is a tubular body, a sufficient amount of tissue can be collected into the inner side of the puncture tube 2. Further, the puncture tube 2 can advance while rotating in the application region and can shred the tissue because the spiral blade 3 is provided in a projecting manner on the inner surface of the puncture tube 2. The spiral blade 3 achieves a function of a lid (i.e., acts as a collecting member to collect the living body tissue) at the distal portion 22 of the puncture tube 2. That is, when the sheath member 1 and/or the puncture tube 2 are to be pulled out from the living body in a state in which tissue is collected in the puncture tube 2, the spiral blade 3 can help prevent the tissue from falling from (i.e., out of) the distal portion 22 of the puncture tube 2.

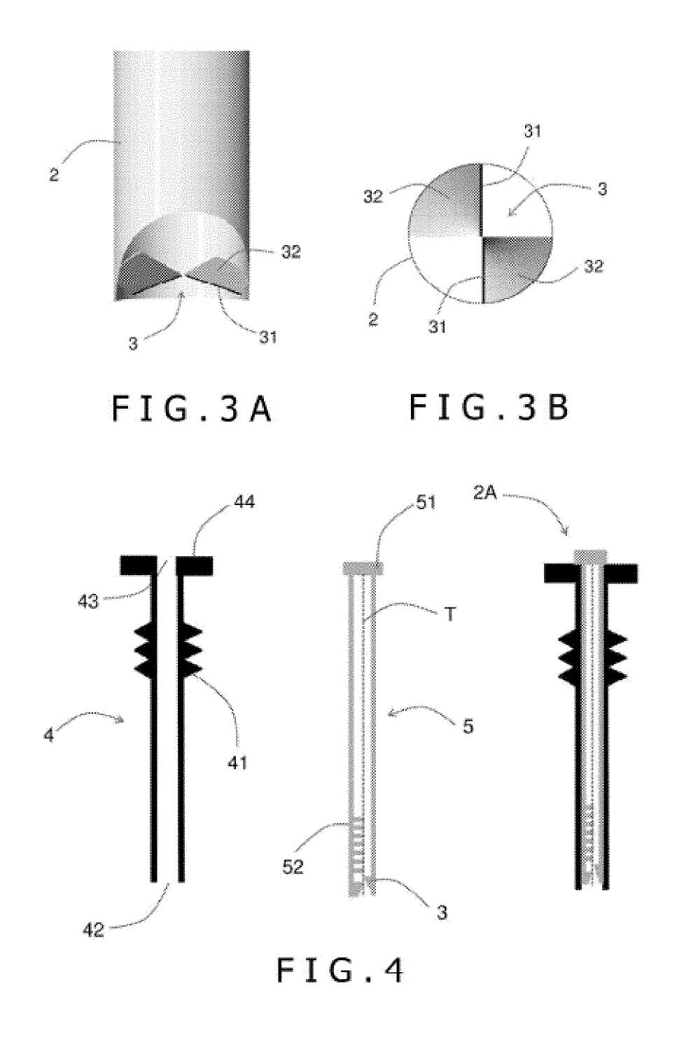

FIGS. 3A and 3B are partial sectional views schematically depicting the spiral blade 3 of the tissue collection device. As depicted in FIG. 3A, the spiral blade 3 includes two plate-shaped members 32 each having a blade 31 at a distal edge thereof. The two plate-shaped members 32 are inclined with respect to a plane perpendicular to the long axis (longitudinal axis) of the puncture tube 2. The blade 31 is disposed such that it is inclined with respect to the plane perpendicular to the long axis of the puncture tube 2 and can cut obliquely into the surface of the application region to shred the tissue of the living body. The spiral blade 3 is provided in a projecting manner on an inner face of a distal portion 22 of the puncture tube 2 (i.e., the spiral blade 3 extends radially inwardly from the inner surface of the puncture tube 2 along a length of the distal portion 22 of the puncture tube 2). The length of the spiral blade 3 in the axial direction is smaller than the length of the puncture tube 2 in the axial direction. In particular, the spiral blade 3 is not provided on the proximal side with respect to the distal portion of the puncture tube 2 (i.e., the spiral blade 3 is only located in the vicinity of the distal end of the puncture tube 2 as shown in FIGS. 1-3) and is not an obstacle when the tissue collected in the puncture tube 2 is to be taken out (i.e., removed from the puncture tube 2). The spiral blade 3 is fixed to the inner face of the puncture tube 2. A support such as an auger screw thus does not occupy the volume of the puncture tube 2, and so a relatively greater amount of tissue can be collected in the puncture tube 2.

FIG. 3B is a view of the puncture tube 2 on which the spiral blade 3 is provided in a projecting manner as viewed from the axial direction (i.e., FIG. 3B shows a cross-section of the puncture tube 2 at the location of the spiral blade 3). The plate-shaped members 32 have a sectoral shape of an angle of 90 degrees as viewed from the axial direction (i.e., the plate-shaped members 32 each are a quarter of a circular disc as shown in FIG. 3B). The two plate-shaped members 32 are provided in a projecting manner at positions opposing to each other (i.e., diametrically opposite) on the inner face of the puncture tube 2. In other words, the two plate-shaped members 32 are disposed such that they draw a double spiral in the puncture tube 2 and can shred the tissue more finely (relatively). The two blades 31 of the two plate-shaped members 32 are disposed such that they cross the diameter (i.e., span across the inner diameter of the puncture tube 2 as shown in FIG. 3B) of the puncture tube 2. When the puncture tube 2 is to be inserted into an application region of a living body while being rotated, the spiral blade 3 is thus likely to cut into the tissue. The tissue collected in the puncture tube 2 can be held with a higher degree of certainty because the two plate-shaped members 32 oppose one another around the inner circumference of the puncture tube 2.

The device according to the first embodiment can thereby collect tissue quantitatively (i.e., by a set or predetermined amount) by a single time operation without depending upon the manipulation of the operator (i.e., manual operation to draw a certain or desired amount of tissue), and therefore, relatively low invasiveness of the living body can be achieved.

The sheath member 1 of the disclosed tissue collective device is not limited to that of the first embodiment but can assume various modifications. For example, the stopper 13 of the sheath member 1 is not limited to such a stopper that utilizes an offset as described above. For example, a flange portion may be provided which can be fixed to an outer face of the sheath member 1 but is slidably movable in the axial direction of the sheath member 1. This flange portion may allow quantitative tissue collection to be achieved by slidably moving the flange portion with reference to such graduations on the outer face of the sheath member 1 in a similar manner to that described above.

The spiral pitch of the spiral blade 3 may be made equal to the thread pitch between the first and second threaded portions 11, 21 of the puncture tube 2 and the sheath member 1. Consequently, it is possible to minimize destruction of the tissue by the spiral blade 3 and advance the spiral blade 3 smoothly into the tissue. The angle of the two plate-shaped members 32 configuring the spiral blade 3 as viewed from the axial direction is not limited to 90 degrees and can be set freely within the range of 1 to 360 degrees or exceeding 360 degrees. In particular, the spiral blade 3 may be a blade in the form of a string, a sectoral blade or a spiral blade of 360 degrees or more. Also the number of such plate-shaped members 32 is not limited to two but can be set freely, for example, from one plate-shaped member to ten or more plate-shaped members. The projection height of the spiral blade 3 in the center direction (i.e., the extension length of the spiral blade 3 inward from the inner surface of the puncture tube 2) may be greater than the radius of the puncture tube 2 to increase the cutting away effect of the tissue.

### Second Embodiment

Now, a second embodiment of a tissue collection device is described in relation to FIG. 4. FIG. 4 is a sectional view schematically depicting the device according to the second embodiment. Note that, in the following description, portions/components that are equivalent to those of the device according to the first embodiment are denoted by the same reference characters and description of these portions/components is omitted.

In the embodiment shown in FIG. 4, a puncture tube 2A includes an inner side tubular body 5 (shown in the center of FIG. 4) and an outer side tubular body 4 (shown on the left side of FIG. 4). The inner side tubular body 5 is fitted for back and forth movement (i.e., proximal and distal movement) in the outer side tubular body 4 as shown in the right side of FIG. 4 (i.e., the inner side tubular body 5 fits within and is movable relative to the outer side tubular body 4). The outer side tubular body 4 has an opening 43 on the proximal side of the outer side tubular body 4 such that the inner side tubular body 5 can be fitted into the outer side tubular body 4 from the proximal side. The outer diameter of the inner side tubular body 5 (distal to the grasping unit 51) is thus smaller than the inner diameter of the outer side tubular body 4 as shown in FIG. 4. The inner side tubular body 5 has a spiral blade 3 provided in a projecting manner on an inner face thereof, and the outer side tubular body 4 has a threaded portion 41 provided in a projecting manner on an outer face thereof. The first threaded portion 11 of the sheath member 1 can be threadedly engaged with the threaded portion 41 of the outer side tubular body 4 in a similar manner as discussed above regarding FIG. 1. The tissue of the living body may thus be collected into the inside of the inner side tubular body 5.

A grasping unit 51 is provided on the proximal side of the inner side tubular body 5. The outer diameter of the grasping unit 51 is greater than the inner diameter of the outer side tubular body 4 so that the grasping unit 51 is prevented from entering the interior of the outer side tubular body 4. Although an operator can grip the grasping unit 51 to slidably move the inner side tubular body 5 in the outer side tubular body 4 (and relative to the outer side tubular body 4), the advancement of the grasping unit 51 is stopped on the proximal side of the outer side tubular body 4. The grasping unit 51 can be removably fixed to an operation unit 44 of the outer side tubular body 4, so that the outer side tubular body 4 and the inner side tubular body 5 can be rotated integrally.

A reduced thickness portion T extending in the axial direction is provided on the inner side tubular body 5. The tissue of the living body can be taken out (i.e., removed or extracted) readily by an operator pulling out the inner side tubular body 5 from the outer side tubular body 4 (i.e., retracting the inner side tubular body 5 in the proximal direction relative to the outer side tubular body 4) and putting a bar-like member or air into the inner side tubular body 5 from the proximal side (or by like means) to push out the tissue to the distal side or dividing (i.e., splitting or opening) the inner side tubular body 5 longitudinally along the reduced thickness portion T. The inner side tubular body 5 is configured from an optically transparent material and has graduations 52 on an outer face of the inner side tubular body 5. Accordingly, the operator can confirm the amount of tissue collected in the inner side tubular body 5 from outside of the inner side tubular body 5. If the tissue collection amount is relatively small (i.e., too small), a sufficient amount of tissue can be taken in by returning the inner side tubular body 5 into the outer side tubular body 4 and rotating the puncture tube 2A in the sheath member 1 again.

Since the device according to the second embodiment can collect tissue quantitatively by a single time operation without depending upon the manipulation of the operator, relatively low invasiveness can be achieved.

The puncture tube 2A is not restricted to the embodiment of FIG. 4, but can assume various modifications. For example, both the inner side tubular body 5 and the outer side tubular body 4 may be configured from an optically transparent material such that the collection amount of tissue can be confirmed only by taking the puncture tube 2A out from the sheath member 1. The sheath member 1 may also be configured from an optically transparent material such that the collection amount of tissue can be confirmed without taking out the puncture tube 2A from the sheath member 1.

### Third Embodiment

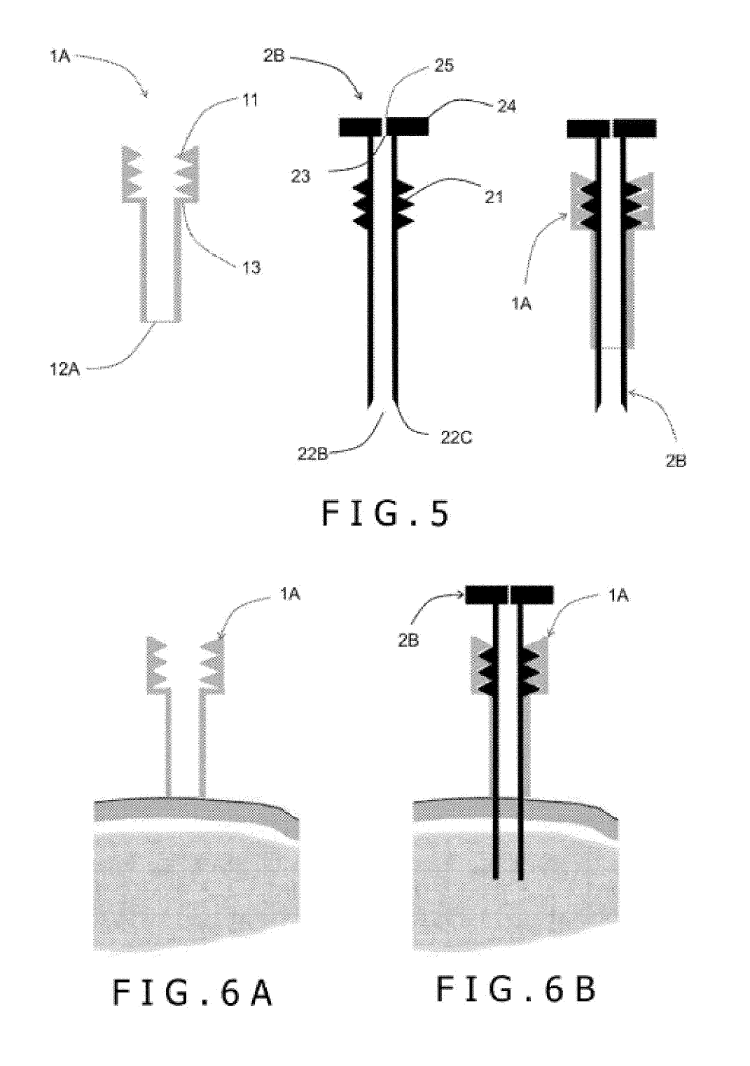

A third embodiment of a tissue collection device is described in relation to FIG. 5. FIG. 5 is a sectional view schematically depicting the device according to the third embodiment. In the following description, the portions/components of the devices according to the first and second embodiments described above are denoted by the same reference characters and description of these portions/components is omitted.

FIG. 5 illustrates that the length of a sheath member 1A (i.e., depicted on the left side of FIG. 5) on the distal side with respect to a first threaded portion 11 (i.e., the length of the sheath member 1A distal to the first threaded portion 11) is smaller than the length of a puncture tube 2B (i.e., depicted in the center of FIG. 5) on the distal side with respect to a second threaded portion 21 (i.e., the length of the puncture tube 2B distal to the second threaded portion 21). A distal portion 12A of the sheath member 1A extends in parallel to a plane perpendicular to the long axis of the sheath member 1A. The distal portion 12A of the sheath member 1A is designed such that it has no blade provided thereon (i.e., the sheath member 1A is devoid of a blade) and, even if the distal portion 12A of the sheath member 1A is pressed against an application region of a living body, the sheath member 1A does not pierce the application region. A distal portion 22B of the puncture tube 2B extends in parallel to a plane perpendicular to the long axis of the puncture tube 2B. The inner face of the puncture tube 2B does not possess a spiral blade provided thereon as shown in FIG. 5, and instead, a blade 22C is provided at a lower end of the puncture tube 2B (namely, at the distal portion 22B). Accordingly, the distal portion 22B of the puncture tube 2B can puncture an application region to perform incision and cutting out of tissue and can then take in (i.e., collect) the tissue into the inner side of the puncture tube 2B. An opening 25 is provided at a proximal portion 23 (proximal end as shown in FIG. 5) of the puncture tube 2B. If the puncture tube 2B is fitted into the sheath member 1A so that the first and second threaded portions 11, 21 threadedly engage with one another, then the distal side of the puncture tube 2B is exposed from (i.e., extends distally beyond) the distal portion 12A of the sheath member 1A (as shown on the right side of FIG. 5).

FIGS. 6A and 6B are sectional views depicting an example of use of the device according to the third embodiment. The distal portion 12A of the sheath member 1A is placed on the surface of an application region (e.g., the skin) of a living body as shown in FIG. 6A. The sheath member 1A does not pierce the application region because the distal portion 12A of the sheath member 1A does not have a blade and is not configured to puncture the skin. The puncture tube 2B is then inserted into the sheath member 1A such that the second threaded portion 21 of the puncture tube 2B and the first threaded portion 11 of the sheath member 1A are threadedly engaged with each other. The operation unit 24 is then operated to rotate the puncture tube 2B. The blade 22C provided at the distal portion 22B of the puncture tube 2B incises the surface of the application region to cut away tissue and collects the cut tissue into the inner side of the puncture tube 2B as shown in FIG. 6B. When the tissue is to be removed from the living body, the inside of the puncture tube 2B is enclosed in a state in which the opening 25 of the puncture tube 2B is closed with a finger or the like, and the puncture tube 2B is pulled out (i.e., retracted proximally) together with the sheath member 1A. By this, the tissue can be extracted from the application region.

The device illustrated in FIG. 5 can thus collect tissue quantitatively (i.e., a set or predetermined amount) by a single time operation without depending upon the manipulation of the operator, and therefore, relatively low invasiveness of the living body can be achieved.

The puncture tube 2B is not limited to any particular embodiment, but can assume various modifications. For example, in addition to the blade at the distal portion 22B of the puncture tube 2B, a spiral blade may be provided in a projecting manner on the inner face of the puncture tube 2B (i.e., the blade may extend radially inward from the inner surface of the puncture tube 2B at a distal portion of the puncture tube 2B). The opening 25 may be openable and closeable such that the inside of the puncture tube 2B is enclosed without depending upon the manipulation by the operator. The puncture tube 2B may include a piston member that can be inserted into the opening 25. The piston member can be slidably moved in the puncture tube 2B to draw up tissue taken in the puncture tube 2B to the proximal portion 23 side (i.e., urge the tissue in the proximal direction), or conversely push out the tissue from the distal portion 22 side of the puncture tube 2B (i.e., urge the tissue in the distal direction).

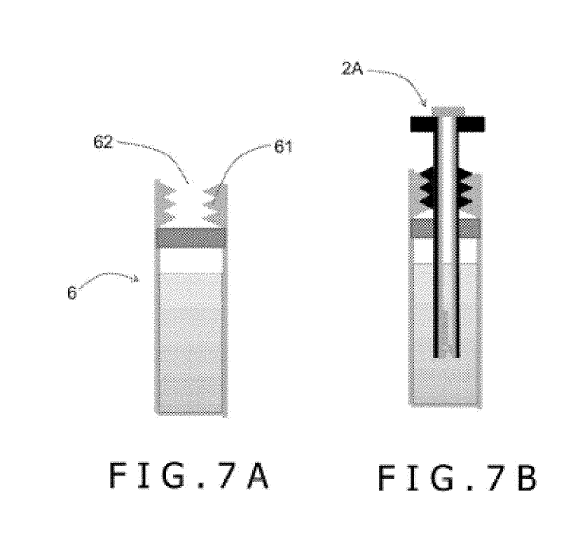

FIGS. 7A and 7B are sectional views schematically depicting a vessel of the present disclosure. As depicted in FIG. 7A, a vessel 6 is a bottomed tubular vessel that can accommodate liquid in the inside of the vessel 6. The liquid is not restricted, and, for example, liquid medium, physiological saline solution, isotonic solution, buffer solution, cryoprotectant and so forth can be used. The vessel 6 has an opening 62, and a third threaded portion 61 is provided in a projecting manner on the inner side of the opening 62 (i.e., the third threaded portion extends radially inward from the inner surface of the vessel 6 as shown in FIG. 7A). The third threaded portion 61 is threadedly engageable with the second threaded portion 21 of the puncture tube 2 in the first embodiment, the second threaded portion 41 of the puncture tube 2A in the second embodiment, and/or the second threaded portion 21 of the puncture tube 2B in the third embodiment. The vessel 6 can accommodate the puncture tube 2, puncture tube 2A and puncture tube 2B therein. FIG. 7B depicts the puncture tube 2A accommodated in the vessel 6.

By threadedly engaging the second threaded portion 41 of the puncture tube 2A with the third threaded portion 61 of the vessel 6, it is possible to threadedly engage the puncture tube 2A with certainty in the vessel 6 to place the vessel 6 into an enclosed state. Tissue is taken in the puncture tube 2A (i.e., the puncture tube 2A has previously extracted tissue from a living body and contains the tissue) and is preserved in a state in which the tissue is immersed in the liquid in the vessel 6. The grasping unit 51 of the inner side tubular body 5 of the puncture tube 2A can be gripped to pull out (i.e., retract proximally) the inner side tubular body 5 from the puncture tube 2A to confirm the state of the tissue in the vessel 6. The state of the tissue can be confirmed simply by this operation. Here, the vessel 6 and the outer side tubular body 4 may be configured from an optically transparent material such that the state of the tissue can be confirmed from the outside of the vessel 6.

While the devices according to various embodiments have been described, the present disclosure is not limited to any particular embodiment(s). Those skilled in the art can design a device having a different configuration or shape by suitably combining the components and the shapes of the devices according to the above description. For example, the blade may be provided selectively at the distal end or distal portion of the sheath member 1, at the distal end or distal portion of the puncture tube 2 or at the distal end or distal portion of the plate-shaped member 32 of the puncture tube 2. A blade of a spiral shape as is used in an auger screw may be provided in a projecting manner on the inner face of the puncture tube 2 or a spiral blade may be attached to a support like an auger screw so as to be slidably movable in the axial direction of the puncture tube 2.

In the present invention, it is possible to replace the components with desired components that can exhibit similar functions or to add desired components.

The detailed description above describes a tissue collection device and a method for using a tissue collection device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A tissue collection device comprising:
a sheath member possessing a distal end and an inner surface surrounding an interior of the sheath member, the sheath member comprising a first threaded portion on the inner surface of the sheath member;
a puncture tube positionable in the interior of the sheath member, the puncture tube being axially movable in distal and proximal directions relative to the sheath member in the interior of the sheath member, the puncture tube possessing an outer surface, an inner surface and a distal end;
the puncture tube comprising a second threaded portion on the outer surface of the puncture tube that threadedly engages with the first threaded portion when the puncture tube is within the interior of the sheath member; and
at least one of the distal end of the sheath member and the distal end of the puncture tube comprising a blade configured to incise tissue from a living body.

2. The tissue collection device according to claim 1, wherein the blade is at the distal end of the puncture tube and is a spiral blade, the spiral blade extending radially inward from the inner surface of the puncture tube.

3. The tissue collection device according to claim 1, wherein
the distal end of the sheath member is a pointed blade to facilitate puncturing and penetration of the living body with the distal end of the sheath member, and
the sheath member comprises graduations on the outer surface of the sheath member to identify a depth of penetration of the distal end of the sheath member into the living body.

4. The tissue collection device according to claim 1, wherein the puncture tube includes an outer side tubular body and an inner side tubular body positionable in the outer side tubular body, the inner side tubular body being axially movable in the distal and proximal directions relative to the outer side tubular body when the inner side tubular body is in the outer side tubular body.

5. The tissue collection device according to claim 4, wherein the inner side tubular body has a groove extending in an axial direction of the inner side tubular body.

6. The tissue collection device according to claim 1, further comprising:
a vessel for accommodating the puncture tube, the vessel having a third threaded portion for threadedly engaging with the second threaded portion.

7. The tissue collection device according to claim 1, wherein the puncture tube is optically transparent.

8. A tissue collection device for collecting tissue in a living body, the tissue collection device comprising:
a sheath member comprising a main body and a first threaded portion, the sheath member extending in an axial direction from a distal end to a proximal end, the first threaded portion of the sheath member being proximal to the main body, the sheath member being a tubular body with the distal and proximal ends being open, the sheath member possessing an outer surface, an inner surface and an inner diameter, the first threaded portion being on one of the outer surface and the inner surface of the sheath member;
a puncture tube positionable in the sheath member, the puncture tube extending in the axial direction from a distal end to a proximal end, the puncture tube comprising a blade at the distal end of the puncture tube and a second threaded portion proximal to the blade, the puncture tube possessing an outer surface, an inner surface and an outer diameter, the second threaded portion being on one of the outer surface and the inner surface of the puncture tube;
the second threaded portion of the puncture tube being configured to threadedly engage the first threaded portion of the sheath member when the puncture tube is positioned in the sheath member so that relative rotation between the sheath member and the puncture tube in one rotational direction axially moves the blade into the tissue in the living body to incise an amount of tissue, and so that relative rotation between the sheath member and the puncture tube in the opposite rotation direction axially moves the blade out of the living body with the incised tissue held at the distal end of the puncture tube; and
the inner diameter of the main body of the sheath member being smaller than the outer diameter of the second threaded portion of the puncture tube, such that the second threaded portion of the puncture tube is prevented from moving distally into the main body of the sheath member.

9. The tissue collection device according to claim 8, wherein the first threaded portion is on the inner surface of the sheath member and the second threaded portion is on the outer surface of the puncture tube.

10. The tissue collection device according to claim 9, wherein
the distal end of the sheath member is a pointed blade to facilitate puncturing and penetration of the living body with the distal end of the sheath member, and
the sheath member comprises graduations on the outer surface of the sheath member to identify a depth of penetration of the distal end of the sheath member into the living body.

11. The tissue collection device according to claim 10, wherein
the first threaded portion possesses a length in the axial direction,
the second threaded portion possesses a length in the axial direction, and
the length of the first threaded portion is equal to the length of the second threaded portion.

12. The tissue collection device according to claim 10, wherein the blade is a spiral blade extending radially inward from the inner surface of the puncture tube.

13. The tissue collection device according to claim 12, wherein the spiral blade comprises two plate-shaped members positioned diametrically opposite to one another on the inner surface of the puncture tube.

14. The tissue collection device according to claim 10, wherein the sheath member comprises a puncturing blade at the distal end of the sheath member.

15. The tissue collection device according to claim 10, wherein the puncture tube comprises an operation member at the proximal end of the puncture tube, the operation member being rotatable to rotate the blade and the second threaded portion of the puncture tube relative to the sheath member.

16. A method for extracting a predetermined amount of tissue from a living body, comprising:
contacting the living body with a distal end of a sheath member;
axially moving a puncture tube positioned inside the sheath member in a distal direction relative to the sheath member to cause a distal end of the puncture tube to contact the tissue in the living body, the axially moving of the puncture tube relative to the sheath member occurring by virtue of a threaded portion of the puncture tube threadedly engaging a threaded portion of the sheath member;
incising the predetermined amount of the tissue in the living body while axially moving the puncture tube in the distal direction;
automatically stopping the axial movement of the puncture tube relative to the sheath member in the distal direction after the predetermined amount of the tissue has been incised; and
extracting the predetermined amount of the tissue from the living body.

17. The method according to claim 16, wherein the contacting of the living body with the distal end of the sheath member comprises puncturing the living body with the sheath member.

18. The method according to claim 16, wherein the contacting of the living body with the distal end of the sheath member comprises contacting an outer surface of the living body with the distal end of the sheath member and maintaining the distal end of the sheath member in contact with the outer surface of the living body during the axial movement of the puncture tube in the distal direction relative to the sheath member to cause the distal end of the puncture tube to contact the tissue in the living body.

19. The method according to claim 16, wherein the extracting of the predetermined amount of the tissue from the living body comprises axially moving the puncture tube in a proximal direction relative to the sheath member.

20. The method according to claim 16, wherein the automatic stopping of the axial movement of the puncture tube comprises preventing the puncture tube from moving distally beyond the threaded portion of the sheath member by virtue of the puncture tube contacting the sheath member when the predetermined amount of the tissue has been incised.
